(19) United States
(12) Patent Application Publication
(10) Pub. No.: US 2024/0360397 A1
(43) Pub. Date: Oct. 31, 2024

(54) SYSTEMS AND METHODS OF IMPEDANCE-BASED BIOMASS SENSING IN FIXED BED BIOREACTORS

(71) Applicant: CORNING INCORPORATED, CORNING, NY (US)

(72) Inventors: Ye Fang, Painted Post, NY (US); Anthony Glenn Frutos, Painted Post, NY (US); Vasiliy Nikolaevich Goral, Painted Post, NY (US); Joel Andrew Schultes, CORNING, NY (US); Elena Ravilievna Streltsova, CORNING, NY (US); Todd Michael Upton, Eliot, ME (US); Jennifer Lynn Weber, Big Flats, NY (US)

(21) Appl. No.: 18/647,679

(22) Filed: Apr. 26, 2024

Related U.S. Application Data
(60) Provisional application No. 63/529,902, filed on Jul. 31, 2023.
(60) Provisional application No. 63/462,761, filed on Apr. 28, 2023.

(51) Int. Cl. C12M 1/12; C12M 1/00; C12M 1/34

(52) U.S. Cl. CPC C12M 25/18; C12M 29/04; C12M 41/36

(57) ABSTRACT

A cell culture bioreactor is disclosed that has a cell culture vessel with an interior reservoir to contain a cell substrate, an inlet fluidly connected to the reservoir, and an outlet fluidly connect to the reservoir. The bioreactor also has a sensor system for measuring impedance within the reservoir. The sensor system includes a pair of electrodes that are spaced apart from each other with at least a portion of the cell substrate disposed between the pair of electrodes. Each electrode of the pair of electrodes includes a plurality of pores to allow at least one of cells and culture media to flow through the electrode.

### CROSS-REFERENCE TO RELATED APPLICATIONS

This application claims the benefit of priority under 35 U.S.C. § 119 of U.S. Provisional Application Ser. No. 63/529,902 filed on Jul. 31, 2023 and U.S. Provisional Application Ser. No. 63/462,761 filed on Apr. 28, 2023, the content of which are relied upon and incorporated herein by reference in their entirety.

### FIELD OF THE DISCLOSURE

This disclosure general relates to systems and methods of monitoring cell cultures in bioreactor systems. In particular, the present disclosure relates to systems and methods for biomass monitoring of a cell culture within a fixed bed bioreactor system using impedance-based sensors within the bioreactor to, among other things, detect cell confluency and culture quality.

### BACKGROUND

In the bioprocessing industry, large-scale cultivation of cells is performed for purposes of the production of hormones, enzymes, antibodies, vaccines, therapeutic proteins, and cell therapies. Cell and gene therapy markets are growing rapidly, with promising treatments moving into clinical trials and quickly toward commercialization. However, one cell therapy dose can require billions of cells or trillions of viruses. As such, being able to provide a large quantity of cell products in a short amount of time is critical for clinical success.

A significant portion of the cells used in bioprocessing are anchorage dependent, meaning the cells need a surface to adhere to for growth and functioning. Traditionally, the culturing of adherent cells is performed on two-dimensional (2D) cell-adherent surfaces incorporated in one of a number of vessel formats, such as T-flasks, petri dishes, cell factories, cell stack vessels, roller bottles, and other multilayered vessels (e.g., the HYPERStack® from Corning Inc.). These approaches can have significant drawbacks, including the difficulty in achieving cellular density high enough to make it feasible for large scale production of therapies or cells.

Alternative methods have been suggested to increase volumetric density of cultured cells. These include microcarrier cultures performed in stir tanks; hollow fiber bioreactors, in which cells may form large three-dimensional aggregates as they proliferate in the interspatial fiber space; and packed-bed bioreactors. In packed-bed or fixed-bed bioreactors, a packed or fixed cell substrate is used to provide a surface for the attachment of adherent cells. Medium is perfused along the surface or through the semi-porous substrate to provide nutrients and oxygen needed for the cell growth. For example, packed bed bioreactor systems that contain a packed bed of support or matrix systems to entrap the cells have been previously disclosed U.S. Pat. Nos. 4,833,083; 5,501,971; and 5,510,262. Packed bed matrices usually are made of porous particles as substrates or non-woven microfibers of polymer.

One of the significant issues with traditional fixed bed biorcactors is the non-uniformity of cell distribution inside the bed. For example, the packed bed can function as a depth filter with cells predominantly trapped at the inlet regions or other regions of relatively low flow and/or high substrate density, resulting in a gradient of cell distribution during the inoculation step. In addition, due to random fiber packaging, flow resistance and cell trapping efficiency of cross sections of the packed bed are not uniform. For example, medium flows fast though the regions with low cell packing density and flows slowly through the regions where resistance is higher due to higher number of entrapped cells. This creates a channeling effect where nutrients and oxygen are delivered more efficiently to regions with lower volumetric cells densities and regions with higher cell densities are being maintained in suboptimal culture conditions.

Another significant drawback of traditional packed bed systems is the inability to efficiently harvest intact viable cells at the end of culture process. Harvesting of cells is important if the end product is cells, or if the bioreactor is being used as part of a “seed train,” where a cell population is grown in one vessel and then transferred to another vessel for further population growth. U.S. Pat. No. 9,273,278 discloses a bioreactor design to improve the efficiency of cell recovery from the packed bed during cells harvesting step. It is based on loosening the packed bed matrix and agitation or stirring of packed bed particles to allow porous matrices to collide and thus detach the cells. However, this approach is laborious and may cause significant cells damage, thus reducing overall cell viability.

In addition, because of the random arrangement of fibers in the traditional packed or fixed bed substrates, it can be difficult for bioreactor users to predict cell culture performance, since the substrate arrangement and/or packing varies between cultures. Monitoring of the cell culture health or progress is also difficult. For example, the presence of the fixed bed itself limits options for effectively monitoring the health of the culture and the biomass production. Furthermore, the packed substrate of traditional fixed bed bioreactors makes efficiently harvesting cells very difficult or impossible, as it is believed that cells are entrapped by the packed bed, which further hampers an understanding of the cell culture performance.

Regardless of the platform used, the earlier stages of process development require users to have information for better understanding of cell behavior, virus production, and culture progress. The upstream bioreactor process development requires the identification of critical parameters and quality features as well as the parameter definition and their connection to the final product. Understanding what these parameters are and how they might scale with higher-density or larger systems is important for process development and efficiency.

Upstream bioprocess production also goes through good manufacturing process (GMP) regulations as well as requirements referred to process analytical technology (PAT). PAT is regarded as a tool for the design, analyses and control of production processes. The final product quality can be ensured through the measurement of process parameters and product characteristics. This can include extensive online culture process monitoring, which provides a useful tool for process characterization and the detection of process changes. Relevant parameters for packed bed bioreactor process characterization and control are pH, temperature, dissolved oxygen or oxygen delivery (DO2), and carbon dioxide (CO2). However, one of the identified drawbacks of packed bed bioreactors is the difficulty in taking substrate samples to directly assess the state of the cells and the overall cell culture progress. Taking substrate samples risk contaminating the entire culture or, in the case of non-uniform platforms, providing misleading or inaccurate data.

In 2004, the U.S. Food & Drug Administration published a guideline to implement process analytical technologies (PAT) in biopharmaceutical processes for process monitoring to gain process understanding and for the control of important process parameters. Bioreactor cell concentration is one of the most important key performance indicators (KPIs) during mammalian cell cultivation processes. In suspension cell culture bioreactors, cell concentration usually is measured offline by taking samples from the reactor or by scalability of linear regression models derived from online capacitance measurements. For example, such measurements are conducted with impedance probes such as BioPAT® ViaMass, Sartorius Stedim Biotech, for single-use applications and a Futura 12 mm Probe, Aber Instruments Ltd., for multi-use applications. Contrary to suspension culture bioreactors, adherent bioreactors built on fixed bed reactor platform are aseptically closed systems that are not suitable for substrate sampling without compromising the sterility. Cells are seeded inside the reactor on a adherent cell substrate. During the culture process, cells proliferate and multiply inside the reactor. Monitoring the rate and degree of cells proliferation is an important part of process control. Radio Frequency Impedance (RFI), for example, provides a unique on-line method for estimating the live cell mass in real time. The RFI technology has been developed to monitor the packed bed by using a sterilizable multi-use 25 mm Flush probe from Aber Instruments (UK) that is placed on top of the substrate of packed bed reactor. In this arrangement, the biomass signal represents the cell concentration on the top carriers in close proximity to the sensor and the assumption has to be made that the cells are evenly distributed, but such even distribution is not always the reality. Thus, it would be preferable to has a sensing technology for detecting biomass throughout the bioreactor.

Fixed bed bioreactors have been increasingly used for scale-up in adherent cell culture. Biomass monitoring is an important tool to design, analyze, and control manufacturing processes of pharmaceuticals when cell culture is involved. For suspension cell culture, biomass monitoring can be achieved using optical or electric approaches. However, for adherent cell culture using fixed bed bioreactors there are not validated approaches or sensors available for online biomass monitoring. Historically, substrate (e.g., glucose) consumption rates have been proposed to be useful for understanding the kinetics of cell growth. However, since glucose consumption rate (GCR) is influenced by many factors besides the viable cell number, there are no established protocols, methods and models to obtain and use effective substrate consumption rates and metabolite accumulation rates for biomass calculation and predictions.

There is a need for bioreactor systems and methods that real-time and aseptic monitoring of cell culture progress, including cell proliferation and density. There is also a need for such monitoring to be conducted in-line and in a representative sample of the cell culture, without disrupting the cell culture.

### SUMMARY

According to embodiments of this disclosure, a cell culture bioreactor is provided, comprising: a cell culture vessel comprising an interior reservoir configured to contain a cell substrate, an inlet fluidly connected to the reservoir, and an outlet fluidly connect to the reservoir; and a sensor system for measuring impedance within the reservoir, the sensor system comprising a pair of electrodes configured to be spaced apart from each other with at least a portion of the cell substrate disposed between the pair of electrodes. Each electrode of the pair of electrodes comprises a plurality of pores configured to allow at least one of cells and culture media to flow through the electrode. The sensor system can further comprise a plurality of pairs of electrodes, each pair of electrodes being configured to be spaced apart from each other with at least a portion of the cell substrate disposed between the pair of electrodes.

According to embodiments, a method of monitoring biomass during a cell culture of cells in a bioreactor is provided, the method comprising: culturing the cells on a fixed bed within the bioreactor using a cell culture medium perfused through the bioreactor; and measuring at least one of impedance and capacitance across at least a portion of the fixed bed during the culturing of the cells.

According to embodiments, a fixed bed cell substrate for culturing adherent cells in a bioreactor, the fixed bed cell substrate comprising: a cell substrate configured for adhering cells thereto for cell culture; and an impedance sensor comprising a pair of electrodes that are spaced apart, wherein at least a portion of the cell substrate is disposed between the pair of electrodes.

### BRIEF DESCRIPTION OF THE DRAWINGS

FIG. 1 is a schematic representation of a cell culture system, according to one or more embodiments.

FIG. 2A is a perspective view of a three-dimensional model of a cell culture substrate, according to one or more embodiments of this disclosure.

FIG. 2B is a two-dimensional plan view of the substrate of FIG. 2A.

FIG. 2C is a cross-section along line A-A of the substrate in FIG. 2B.

FIG. 3 is a schematic representation of a bioreactor fixed bed section that contains two electrodes for impedance-based biomass sensing of the fixed bed, according to embodiments.

FIG. 4 is a schematic representation of a bioreactor fixed bed with multiple impedance-base sensor section, according to embodiments.

### DETAILED DESCRIPTION

Various embodiments of the disclosure will be described in detail with reference to drawings, if any. Reference to various embodiments does not limit the scope of the invention, which is limited only by the scope of the claims attached hereto. Additionally, any examples set forth in this specification are not limiting and merely set forth some of the many possible embodiments of the claimed invention.

Embodiments of this disclosure include systems and methods for monitoring, measuring, and/or predicting the density and/or proliferation of adherent cells inside a fixed bed bioreactor. Embodiments do not require access to samples of the bioreactor cell substrate or fixed bed, nor do they require opening the bioreactor of perfusion loop to the exterior environment (and thus risking contamination of the culture). Rather, embodiments disclosed herein enable, real-time, inline, and aseptic monitoring of a cell culture within a bioreactor. In addition, embodiments of this disclosure enable monitoring the cell culture in different regions or zones of the fixed bed. The embodiments described herein are scalable across all ranges of bioreactor systems, and can be used from process development to production scale systems. According embodiments, fixed bed bioreactor systems, as well as fixed bed cell substrates themselves, are provided with one or more sensors built into the fixed bed. These sensors can be integral components of an aseptic bioreactor system.

Embodiments of this disclosure also include methods of monitoring and/or predicting biomass (e.g., cell density and/or proliferation) within a fixed bed reactor using one or more sensors in one or more zones within a fixed bed. By distributing multiple sensors in multiple zones of the fixed bed, it is possible to monitor cell distribution and proliferation uniformity through the fixed bed. The advantages of embodiments disclosed include the ability to actively monitor the bioreactor or cell culture in real time without the need to perform physical sampling of the fixed bed material for offline analysis. Continuous monitoring of the cell culture allows end users to actively adjust the bioprocess steps that are dependent on the progression of culture processes inside the fixed bed bioreactor, and to identify the timing of positive or negative changes in the state of the cell culture to help pinpoint process parameters in need of changing and optimization. The ability to characterize and log the progression of a cell culture also allows end users to monitor and record batch-to-batch consistency of the process. This type of tracking of progression and consistency among cell culture runs can be incredibly advantageous.

In conventional large-scale cell culture bioreactors, different types of packed-bed bioreactors have been used. Usually, these cell culture beds contain porous matrices to retain adherent or suspension cells, and to support growth and proliferation. Packed-bed matrices provide high surface area to volume ratios, so cell density can be higher than in the other systems. However, the packed bed often functions as a depth filter, where cells are physically trapped or entangled in fibers of the matrix. Thus, because of linear flow of the cell inoculum through the packed bed, cells are subject to heterogeneous distribution inside the packed bed, leading to variations in cell density through the depth or width of the packed bed. For example, cell density may be higher at the inlet region of a bioreactor and significantly lower nearer to the outlet of the bioreactor. In another example, non-uniformities in the packed bed create a channeling effect in which cell culture media preferentially flows in certain areas of the bed while be restricted from reaching other areas of the bed, again leading to non-uniform cell distribution and nonuniform or inconsistent medium or nutrient distribution. This non-uniform distribution of the cells inside of the packed bed significantly hinders scalability and predictability of such bioreactors in bioprocess manufacturing, and can even lead to reduced efficiency in terms of growth of cells or viral vector production per unit surface area or volume of the packed bed.

Another problem encountered in packed bed bioreactors disclosed in prior art is the channeling effect, described above. Due to random nature of packed substrate material and/or random arrangements of nonwoven fibers, the local fiber density at any given cross section of the packed bed is not uniform. Medium flows quickly in the regions with low fiber density (high bed permeability) and much slower in the regions of high fiber density (lower bed permeability). The resulting non-uniform media perfusion across the packed bed creates the channeling effect, which manifests itself as significant nutrient and metabolite gradients that negatively impact overall cell culture and bioreactor performance. Cells located in the regions of low media perfusion will starve and very often die from the lack of nutrients or metabolite poisoning. Cell harvesting is yet another problem encountered when bioreactors packed with non-woven fibrous scaffolds are used. Due to packed-bed functions as depth filter, cells that are released at the end of cell culture process are entrapped inside the packed bed, and cell recovery is very low. This significantly limits utilization of such bioreactors in bioprocesses where live cells are the products. Thus, the non-uniformity leads to areas with different exposure to flow and shear, effectively reducing the usable cell culture area, causing non-uniform culture, and interfering with transfection efficiency and cell release.

To address these and other problems of existing cell culture solutions, embodiments of the present disclosure provide bioreactor systems, fixed bed cell substrates, and methods of using such bioreactor systems and substrates that enable efficient and high-yield cell culturing for anchorage-dependent cells and production of cell products (e.g., proteins, antibodies, viral particles). Embodiments include a porous cell-culture matrix made from an ordered and regular array of porous substrate material that enables uniform cell seeding and media/nutrient perfusion, as well as efficient cell harvesting. Embodiments also enable scalable cell-culture solutions with substrates and bioreactors capable of seeding and growing cells and/or harvesting cell products from a process development scale to a full production size scale, without sacrificing the uniform performance of the embodiments. For example, in some embodiments, a bioreactor can be easily scaled from process development scale to product scale with comparable viral genome per unit surface area of substrate (VG/cm²) across the production scale. The harvestability and scalability of the embodiments herein enable their use in efficient seed trains for growing cell populations at multiple scales on the same cell substrate. In addition, the embodiments herein provide a cell culture matrix having a high surface area that, in combination with the other features described, enables a high yield cell culture solution. In some embodiments, for example, the cell culture substrate and/or bioreactors discussed herein can produce 10¹⁶ to 10¹⁸ viral genomes (VG) per batch.

In addition, the embodiments disclosed herein enable not only cell attachment and growth to a cell culture substrate, but also the viable harvest of cultured cells. The inability to harvest viable cells is a significant drawback in current platforms, and it leads to difficulty in building and sustaining a sufficient number of cells for production capacity. According to an aspect of embodiments of this disclosure, it is possible to harvest viable cells from the cell culture substrate, including between 80% to 100% viable, or about 85% to about 99% viable, or about 90% to about 99% viable. For example, of the cells that are harvested, at least 80% are viable, at least 85% are viable, at least 90% are viable, at least 91% are viable, at least 92% are viable, at least 93% are viable, at least 94% are viable, at least 95% are viable, at least 96% are viable, at least 97% are viable, at least 98% are viable, or at least 99% are viable. Cells may be released from the cell culture substrate using, for example, trypsin, TrypLE, or Accutase.

According to one or more embodiments, a cell culture bioreactor can include a cell substrate within the bioreactor vessel. The substrate can be deployed in a packed bed or fixed bed bioreactor configuration, or in other configurations within a three-dimensional culture chamber of the bioreactor vessel. Due to contamination concerns, the vessel can be a single-use vessel that can be disposed of after use.

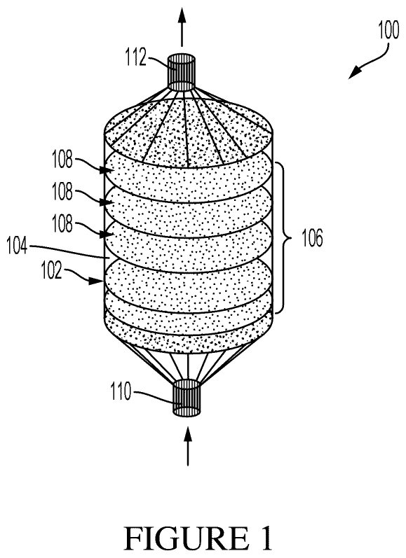

A cell culture bioreactor system is provided, according to one or more embodiments, in which the cell culture substrate is used within an interior reservoir or culture chamber of a bioreactor vessel. FIG. 1 shows an example of a cell culture system 100 that includes a bioreactor vessel 102 having an interior reservoir 104 in the interior of the bioreactor vessel 102. Within the interior reservoir 104 is a fixed bed cell culture substrate 106 that is made from a stack of cell substrate layers 108. The substrate layers 108 are stacked with the first or second major side of a substrate layer facing a first or second major side of an adjacent substrate layer. The bioreactor vessel 100 has an inlet 110 at one end for the input of media, cells, and/or nutrients into the interior reservoir 104, and an outlet 112 at the opposite end for removing media, cells, or cell products from the interior reservoir 104. By allowing stacking of substrate layers in this way, the system can be easily scaled up without negative impacts on cell attachment and proliferation, due to the defined structure and efficient fluid flow through the cell substrate material. While the vessel 100 may generally be described as having an inlet 110 and an outlet 112, some embodiments may use one or both of the inlet 110 and outlet 112 for flowing media, cells, or other contents both into and out of the interior reservoir 104. For example, inlet 110 may be used for flowing media or cells into the culture chamber 104 during cell seeding, perfusion, or culturing phases, but may also be used for removing one or more of media, cells, or cell products through the inlet 110 in a harvesting phase. Thus, the terms “inlet” and “outlet” are not intended to restrict the function of those openings.

In FIG. 1, the bulk flow direction is in a direction from the inlet 110 to the outlet 112, and, in this example, the first and second major sides of the substrate layers 108 are perpendicular to the bulk flow direction. However, embodiments include a fixed bed with a stack of substrates that have first and second sides that are parallel to a bulk flow direction, or a fixed bed having a cylindrical roll of one or more substrate layers. Thus, the substrate of embodiments of this disclosure can be employed in multiple configurations. The fixed bed can be sized and shaped to fill the interior reservoir defined by the culture chamber so that the interior reservoir is filled with cell growth surfaces to maximize efficiency in terms of cells per unit volume.

According to embodiments of this disclosure, the fixed bed cell substrate includes a structurally defined surface area for adherent cells to attach and proliferate that has good mechanical strength and forms a highly uniform multiplicity of interconnected fluidic networks when assembled in a packed bed or other bioreactor. In particular embodiments, a mechanically stable, non-degradable woven mesh can be used as the substrate to support adherent cell production. The cell culture matrix disclosed herein supports attachment and proliferation of anchorage dependent cells in a high volumetric density format. Uniform cell seeding of such a matrix is achievable, as well as efficient harvesting of cells or other products of the bioreactor. In addition, the embodiments of this disclosure support cell culturing to provide uniform cell distribution during the inoculation step and achieve a confluent monolayer or multilayer of adherent cells on the disclosed substrate, and can avoid formation of large and/or uncontrollable 3D cellular aggregates with limited nutrient diffusion and increased metabolite concentrations. Thus, the matrix eliminates diffusional limitations during operation of the bioreactor. In addition, the matrix enables easy and efficient cell harvest from the bioreactor. The structurally defined matrix of one or more embodiments enables complete cell recovery and consistent cell harvesting from the packed bed of the bioreactor.

In contrast to existing cell culture substrates used in cell culture bioreactors (i.e., non-woven substrates of randomly ordered fibers), embodiments of this disclosure include a cell culture substrate having a defined and ordered structure. The defined and order structure allows for consistent and predictable cell culture results. In addition, the substrate has an open porous structure that prevents cell entrapment and enables uniform flow through the packed bed. This construction enables improved cell seeding, nutrient delivery, cell growth, and cell harvesting. According to one or more particular embodiments, the matrix is formed with a substrate material having a thin, sheet-like construction having first and second sides separated by a relatively small thickness, such that the thickness of the sheet is small relative to the width and/or length of the first and second sides of the substrate. In addition, a plurality of holes or openings are formed through the thickness of the substrate. The substrate material between the openings is of a size and geometry that allows cells to adhere to the surface of the substrate material as if it were approximately a two-dimensional (2D) surface, while also allowing adequate fluid flow around the substrate material and through the openings. In some embodiments, the substrate is a polymer-based material, and can be formed as a molded polymer sheet; a polymer sheet with openings punched through the thickness; a number of filaments that are fused into a mesh-like layer; a 3D-printed substrate; or a plurality of filaments that are woven into a mesh layer. The physical structure of the matrix has a high surface-to-volume ratio for culturing anchorage dependent cells. According to various embodiments, the matrix can be arranged or packed in a bioreactor in certain ways discussed here for uniform cell seeding and growth, uniform media perfusion, and efficient cell harvest.

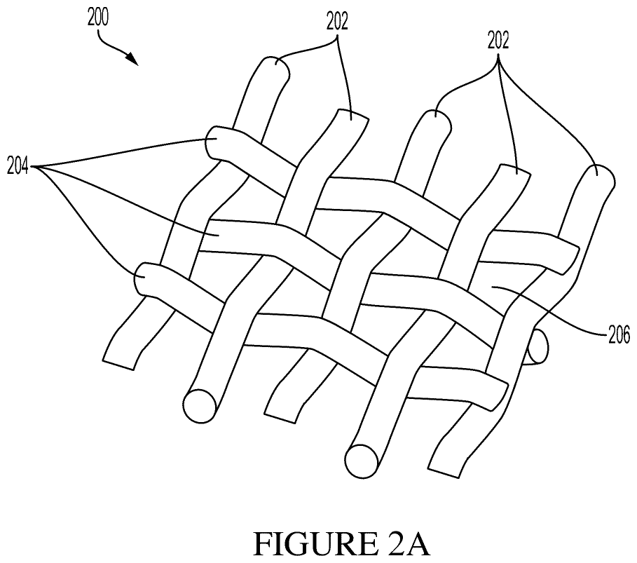

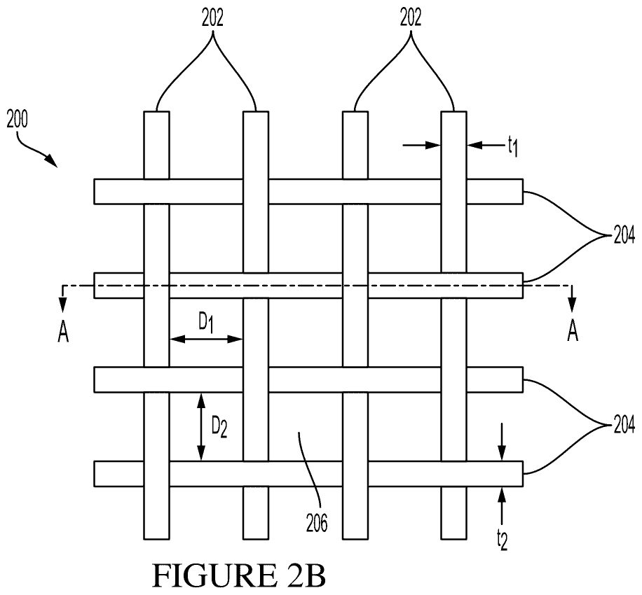

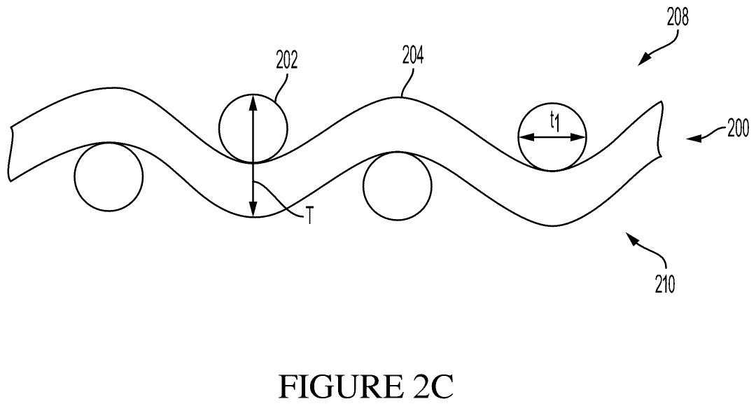

FIGS. 2A and 2B show a three-dimensional (3D) perspective view and a two-dimensional (2D) plan view, respectively, of a cell culture substrate 200, according to an example of one or more embodiments of this disclosure. The cell culture substrate 200 is a woven mesh layer made of a first plurality of fibers 202 running in a first direction and a second plurality of fibers 204 running in a second direction. The woven fibers of the substrate 200 form a plurality of openings 206, which can be defined by one or more widths or diameters (e.g., D₁, D₂). The size and shape of the openings can vary based on the type of weave (e.g., number, shape and size of filaments; angle between intersecting filaments, etc.). A woven mesh may be characterized as, on a macro-scale, a two-dimensional sheet or layer. However, a close inspection of a woven mesh reveals a three-dimensional structure due to the rising and falling of intersecting fibers of the mesh. Thus, as shown in FIG. 2C, a thickness T of the woven mesh 200 may be thicker than the thickness of a single fiber (e.g., t₁). As used herein, the thickness T is the maximum thickness between a first side 208 and a second side 210 of the woven mesh. Without wishing to be bound by theory, it is believed that the three-dimensional structure of the substrate 200 is advantageous as it provides a large surface area for culturing adherent cells, and the structural rigidity of the mesh can provide a consistent and predictable cell culture matrix structure that enables uniform fluid flow.

In FIG. 2B, the openings 206 have a diameter D₁, defined as a distance between opposite fibers 202, and a diameter D₂, defined as a distance between opposite fibers 204. D₁ and D₂ can be equal or unequal, depending on the weave geometry. Where D₁ and D₂ are unequal, the larger can be referred to as the major diameter, and the smaller as the minor diameter. In some embodiments, the diameter of an opening may refer to the widest part of the opening. Unless otherwise specified, the opening diameter, as used herein, will refer to a distance between parallel fibers on opposite sides of an opening.

A given fiber of the plurality of fibers 202 has a thickness t₁, and a given fiber of the plurality of fibers 204 has a thickness t₂. In the case of fibers of round cross-section, as shown in FIG. 2A, or other three-dimensional cross-sections, the thicknesses t₁ and t₂ are the maximum diameters or thicknesses of the fiber cross-section. According to some embodiments, the plurality of fibers 202 all have the same thickness t₁, and the plurality of fiber 204 all have the same thickness t₂. In addition, t₁ and t₂ may be equal. However, in one or more embodiments, t₁ and t₂ are not equal such as when the plurality of fibers 202 are different from the plurality of fiber 204. In addition, each of the plurality of fibers 202 and plurality of fibers 204 may contain fibers of two or more different thicknesses (e.g., t₁ₐ, t_{1b}, etc., and t₂ₐ, t_{2b}, etc.). According to embodiments, the thicknesses t₁ and t₂ are large relative to the size of the cells cultured thereon, so that the fibers provide an approximation of a flat surface from the perspective of the cell, which can enable better cell attachment and growth as compared to some other solutions in which the fiber size is small (e.g., on the scale of the cell diameter). Due to three-dimensional nature of woven mesh, as shown in FIGS. 2A-2C, the 2D surface area of the fibers available for cell attachment and proliferation exceeds the surface area for attachment on an equivalent planar 2D surface.

In one or more embodiments, a fiber may have a diameter in a range of about 20 μm to about 1000 μm; about 100 μm to about 750 μm; about 125 μm to about 600 μm; about 150 μm to about 500 μm; about 200 μm to about 400 μm; about 200 μm to about 300 μm; or about 150 μm to about 300 μm. On a microscale level, due to the scale of the fiber compared to the cells (e.g., the fiber diameters being larger than the cells), the surface of monofilament fiber is presented as an approximation of a 2D surface for adherent cells to attach and proliferate. Fibers can be woven into a mesh with openings ranging from about 100 μm×100 μm to about 1000 μm×1000 μm. In some embodiments, the opening may have a diameter of about 50 μm to about 1000 μm; about 100 μm to about 750 μm; about 125 μm to about 600 μm; about 150 μm to about 500 μm; about 200 μm to about 400 μm; or about 200 μm to about 300 μm. These ranges of the filament diameters and opening diameters are examples of some embodiments, but are not intended to limit the possible feature sizes of the mesh according to all embodiments. The combination of fiber diameter and opening diameter is chosen to provide efficient and uniform fluid flow through the substrate when, for example, the cell culture matrix is comprises a number of adjacent mesh layers (e.g., a stack of individual layers or a rolled mesh layer).

Factors such as the fiber diameter, opening diameter, and weave type/pattern will determine the surface area available for cell attachment and growth. In addition, when the cell culture matrix includes a stack, roll, or other arrangement of overlapping substrate, the packing density of the cell culture matrix will impact the surface area of the packed bed matrix. Packing density can vary with the packing thickness of the substrate material (e.g., the space needed for a layer of the substrate). For example, if a stack of cell culture matrix has a certain height, each layer of the stack can be said to have a packing thickness determined by dividing the total height of the stack by the number of layers in the stack. The packing thickness will vary based on fiber diameter and weave, but can also vary based the alignment of adjacent layers in the stack. For instance, due to the three-dimensional nature of a woven layer, there is a certain amount of interlocking or overlapping that adjacent layers can accommodate based on their alignment with one another. In a first alignment, the adjacent layers can be tightly nestled together, but in a second alignment, the adjacent layers can have zero overlap, such as when the lower-most point of the upper layer is in direct contact with the upper-most point of the lower layer. It may be desirable for certain applications to provide a cell culture matrix with a lower density packing of layers (e.g., when higher permeability is a priority) or a higher density of packing (e.g., when maximizing substrate surface area is a priority). According to one or more embodiments, the packing thickness can be from about 20 μm to about 1000 μm; about 100 μm to about 750 μm; about 125 μm to about 600 μm; about 150 μm to about 500 μm; about 200 μm to about 400 μm; about 200 μm to about 300 μm.

The above structural factors can determine the surface area of a cell culture matrix, whether of a single layer of cell culture substrate or of a cell culture matrix having multiple layers of substrate). For example, in a particular embodiment, a single layer of woven mesh substrate having a circular shape and diameter of 6 cm can have an effective surface area of about 68 cm². The “effective surface area,” as used herein, is the total surface area of fibers in a portion of substrate material that is available for cell attachment and growth. Unless stated otherwise, references to “surface area” refer to this effective surface area.

The substrate mesh can be fabricated from monofilament or multifilament fibers of polymeric materials compatible in cell culture applications, including, for example, polystyrene, polyethylene terephthalate, polycarbonate, polyvinylpyrrolidone, polybutadiene, polyvinylchloride, polyethylene oxide, polypyrroles, and polypropylene oxide. Mesh substrates may have a different patterns or weaves, including, for example knitted, warp-knitted, or woven (e.g., plain weave, twilled weave, dutch weave, five needle weave).

The surface chemistry of the mesh filaments may need to be modified to provide desired cell adhesion properties. Such modifications can be made through the chemical treatment of the polymer material of the mesh or by grafting cell adhesion molecules to the filament surface. Alternatively, meshes can be coated with thin layer of biocompatible hydrogels that demonstrate cell adherence properties, including, for example, collagen or Matrigel®. Alternatively, surfaces of filament fibers of the mesh can be rendered with cell adhesive properties through the treatment processes with various types of plasmas, process gases, and/or chemicals known in the industry. In one or more embodiments, however, the mesh is capable of providing an efficient cell growth surface without surface treatment.

In embodiments, a cell substrate can be packed in a cylindrical roll format within the bioreactor. The scalability of such a fixed bed bioreactor can be achieved by increasing the overall length of the substrate strip and its height. The amount of substrate used in this cylindrical roll configuration can vary based on the desired packing density of the fixed bed. For example, the cylindrical rolls can be densely packed in a tight roll or loosely packed in a loose roll. The density of packing will often be determined by the required cell culture substrate surface area required for a given application or scale.

By using a structurally defined culture matrix of sufficient rigidity, high-flow-resistance uniformity across the matrix or packed bed is achieved. According to various embodiments, the substrate fixed bed can be deployed in monolayer or multilayer formats. This flexibility eliminates diffusional limitations and provides uniform delivery of nutrients and oxygen to cells attached to the matrix. In addition, the open matrix lacks any cell entrapment regions in the packed bed configuration, allowing for complete cell harvest with high viability at the end of culturing. The matrix also delivers packaging uniformity for the fixed bed, and enables direct scalability from process development units to large-scale industrial bioprocessing unit. The ability to directly harvest cells from the packed bed eliminates the need of resuspending a matrix in a stirred or mechanically shaken vessel, which would add complexity and can inflict harmful shear stresses on the cells. Further, the high packing density of the cell culture matrix yields high bioprocess productivity in volumes manageable at the industrial scale.

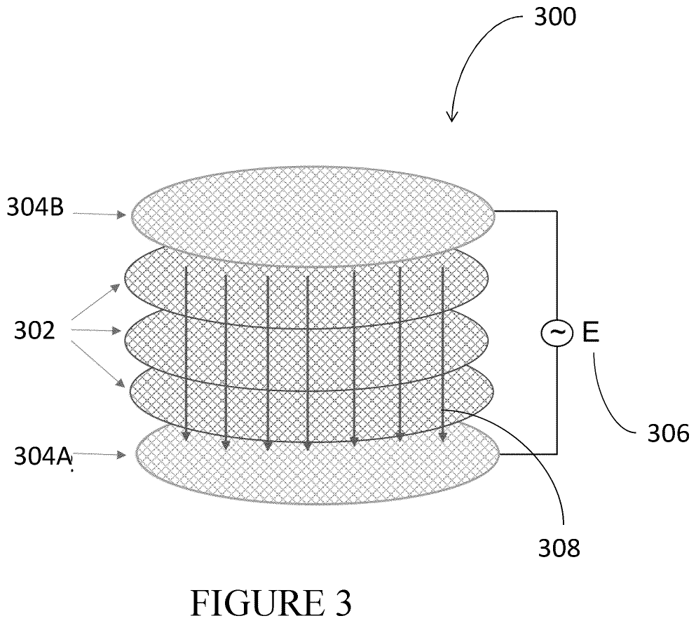

FIG. 3 shows an example of embodiments of a bioreactor fixed bed section 300 that contains two electrodes for impedance-based biomass sensing of the fixed bed. The fixed bed section 300 includes at least a portion of the fixed bed substrate 302, which in this example includes 3 layers of cell substrate material. The two electrodes 304A, 304B are spaced apart from each other with the cell substrate layers 302 occupying the space separating the electrodes 304A, 304B. With application of a voltage 306, an electric field 308 is created by the electrodes 304A, 304B. Using impedance spectroscopy, the resistance (impedance) of an electrochemical system is measured under the application of the applied voltage. The electrodes 304A, 304B can be made from the same material as the cell substrate 302, but with the addition of a conductive coating, such as gold. By using the same material, or at least an electrode with the approximately same structure (e.g., porosity and/or structural arrangement), the fixed bed can perform as design with minimal or no negative impact from the presence of the electrodes 304A, 304B.

The cell substrate (e.g., layers of mesh) is considered part of the electro chemical system. To enable impedance spectroscopy measurements in a fixed bed bioreactor, embodiments of this disclosure include using planar mesh-like electrodes within the fixed bed. The electrodes effectively act as substrate replacement layers and have porosity to allow fluid flow through the electrodes and otherwise not interrupt the flow and progress of the cell culture. When cells are cultured on top of the gold electrodes, and on top of the substrate located between the electrodes, they alter the current pathways due to the insulating properties of the cell plasma membranes. The measured impedance increases with cell growth until it and reaches a plateau as the cells form a confluent monolayer and stop further growth on top of the electrodes and adherent substrate. The combination of low void volume of packed bed and dielectric properties of bulk cells growth substrate (3) will reduce the contribution of medium to impedance. By varying the amount of growth substrate layers placed between sensing electrodes, the system can be fine-tuned to obtain the best signal-to-noise ratio.

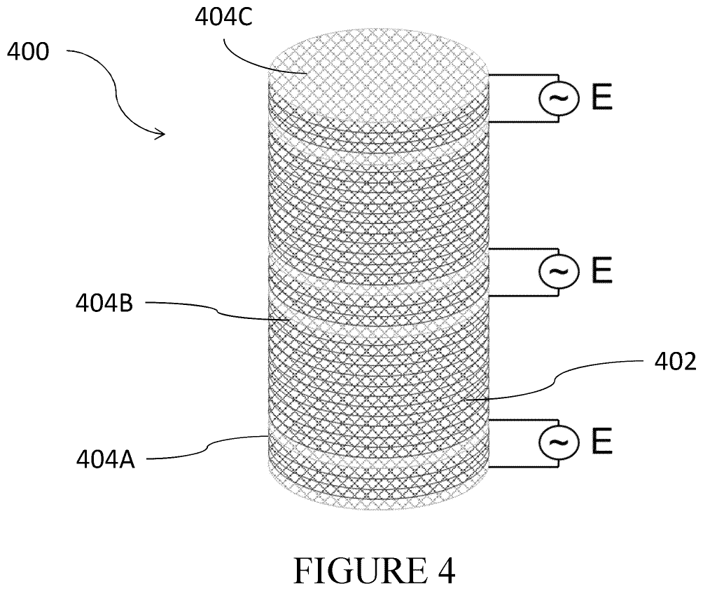

In addition, it is possible to monitor cell distribution uniformity and proliferation in different zones of the packed bed reactor by placing additional pairs of sensing electrodes throughout fixed bed. For example, FIG. 4 shows a fixed bed 400 with a plurality of stacked substrate layers 402, and multiple pairs of electrodes 404A, 404B, and 404C. The multiple pairs of electrodes can be strategically placed through the fixed bed. For example, one pair 404A is shown at a bottom of the fixed bed, one pair 404B in the center, and one pair 404C at the top of the fixed bed, to detect potential variations in cell density across different portions of the bioreactor fixed bed.

The cell culture matrix can be arranged in multiple configurations within the culture chamber depending on the desired system. For example, in one or more embodiments, the system includes one or more layers of the substrate with a width extending across the width of a defined cell culture space in the culture chamber. Multiple layers of the substrate may be stacked in this way to a predetermined height. The substrate layers may be arranged such that the first and second sides of one or more layers are perpendicular to a bulk flow direction of culture media through the defined culture space within the culture chamber, or the first and second sides of one or more layers may be parallel to the bulk flow direction. In one or more embodiments, the cell culture matrix includes one or more substrate layers at a first orientation with respect to the bulk flow, and one or more other layers at a second orientation that is different from the first orientation. For example, various layers may have first and second sides that are parallel or perpendicular to the bulk flow direction, or at some angle in between.

In one or more embodiments, the cell culture system includes a plurality of discrete pieces of the cell culture substrate in a packed bed configuration, where the length and or width of the pieces of substrate are small relative to the culture chamber. As used herein, the pieces of substrate are considered to have a length and/or width that is small relative to the culture chamber when the length and/or width of the piece of substrate is about 50% or less of the length and/or width of the culture space. Thus, the cell culture system may include a plurality of pieces of substrate packed into the culture space in a desired arrangement. The arrangement of substrate pieces may be random or semi-random, or may have a predetermined order or alignment, such as the pieces being oriented in a substantially similar orientation (e.g., horizontal, vertical, or at an angle between 0° and 90° relative to the bulk flow direction).

The “defined culture space,” as used herein, refers to a space within the culture chamber occupied by the cell culture matrix and in which cell seeding and/or culturing is to occur. The defined culture space can fill approximately the entirety of the culture chamber, or may occupy a portion of the space within the culture chamber. As used herein, the “bulk flow direction” is defined as a direction of bulk mass flow of fluid or culture media through or over the cell culture matrix during the culturing of cells, and/or during the inflow or outflow of culture media to the culture chamber.

In some embodiments of this disclosure, there is only a single bulk flow direction within the defined culture space, the packed bed, and/or the bioreactor vessel, such that the liquid or media flow proceeds in predominately one direction from the bioreactor inlet through the packed bed to the bioreactor outlet. The liquid or media flow is uninterrupted by any complicated flow paths within the packed bed space and proceeds through the packed bed in predominately one direction. This avoids complicating flow paths used in some conventional bioreactors where flow spacers, separators, or channels are used to help distribute cell culture media through a cell culture substrate, often because of the inherent non-uniformity of the bioreactor or cell culture substrate. However, in embodiments of the current disclosure, such complicated flow paths are not necessary, and the media flow can be maintained in a single direction from the inlet of the bioreactor to the outlet of the bioreactor. The foregoing is not intended to preclude the use of flow distributor plates at the inlet and outlets of the bioreactor plate, which can be used to distribute fluid across a width of the bioreactor vessel and/or control pressure differentials within the reactor, but do not otherwise affect the bulk flow direction through the packed bed and/or within the cell culture space within the bioreactor vessel interior.

The packed bed cell culture matrix of one or more embodiments can include a substrate material constructed to have a uniform and ordered porous structure. The substrate may be referred to as a “structurally defined” substrate meaning that the substrate has a physical structure that is non-random, but instead is ordered according to defined parameters. In one or more embodiments, the structurally defined substrate includes a plurality of openings defining a porosity of the substrate, the plurality of openings being arrayed in a regular or uniform pattern in each substrate piece or layer. In one or more embodiments, the packed bed cell culture substrate may include a woven cell culture mesh substrate without any other form of cell culture substrate disposed in or interspersed with the cell culture matrix. That is, the woven cell culture mesh substrate of embodiments of this disclosure are effective cell culture substrates without requiring the type of irregular, non-woven substrates used in existing solution. This enables cell culture systems of simplified design and construction, while providing a high-density cell culture substrate with the other advantages discussed herein related to flow uniformity, harvestability, etc.

In one or more embodiments, a matrix is provided with a structurally defined surface area for adherent cells to attach and proliferate that has good mechanical strength and forms a highly uniform multiplicity of interconnected fluidic networks when assembled in a packed bed or other bioreactor. In particular embodiments, a mechanically stable, non-degradable woven mesh can be used as the substrate to support adherent cell production. The cell culture matrix disclosed herein supports attachment and proliferation of anchorage dependent cells in a high volumetric density format. Uniform cell seeding of such a matrix is achievable, as well as efficient harvesting of cells or other products of the bioreactor. In addition, the embodiments of this disclosure support cell culturing to provide uniform cell distribution during the inoculation step and achieve a confluent monolayer or multilayer of adherent cells on the disclosed matrix, and can avoid formation of large and/or uncontrollable 3D cellular aggregates with limited nutrient diffusion and increased metabolite concentrations. Thus, the matrix eliminates diffusional limitations during operation of the bioreactor. In addition, the matrix enables easy and efficient cell harvest from the bioreactor. The structurally defined matrix of one or more embodiments enables complete cell recovery and consistent cell harvesting from the packed bed of the bioreactor.

By using a structurally defined culture matrix of sufficient rigidity, high-flow-resistance uniformity across the matrix or packed bed is achieved. According to various embodiments, the matrix can be deployed in monolayer or multilayer formats. This flexibility eliminates diffusional limitations and provides uniform delivery of nutrients and oxygen to cells attached to the matrix. In addition, the open matrix lacks any cell entrapment regions in the packed bed configuration, allowing for complete cell harvest with high viability at the end of culturing. The matrix also delivers packaging uniformity for the packed bed, and enables direct scalability from process development units to large-scale industrial bioprocessing unit. The ability to directly harvest cells from the packed bed eliminates the need of resuspending a matrix in a stirred or mechanically shaken vessel, which would add complexity and can inflict harmful shear stresses on the cells. Further, the high packing density of the cell culture matrix yields high bioprocess productivity in volumes manageable at the industrial scale.

In contrast to existing cell culture substrates used in cell culture bioreactors (i.e., non-woven substrates of randomly ordered fibers), embodiments of this disclosure include a cell culture substrate having a defined and ordered structure. The defined and order structure allows for consistent and predictable cell culture results. In addition, the substrate has an open porous structure that prevents cell entrapment and enables uniform flow through the packed bed. This construction enables improved cell seeding, nutrient delivery, cell growth, and cell harvesting. According to one or more particular embodiments, the matrix is formed with a substrate material having a thin, sheet-like construction having first and second sides separated by a relatively small thickness, such that the thickness of the sheet is small relative to the width and/or length of the first and second sides of the substrate. In addition, a plurality of holes or openings are formed through the thickness of the substrate. The substrate material between the openings is of a size and geometry that allows cells to adhere to the surface of the substrate material as if it were approximately a two-dimensional (2D) surface, while also allowing adequate fluid flow around the substrate material and through the openings. In some embodiments, the substrate is a polymer-based material, and can be formed as a molded polymer sheet; a polymer sheet with openings punched through the thickness; a number of filaments that are fused into a mesh-like layer; a 3D-printed substrate; or a plurality of filaments that are woven into a mesh layer. The physical structure of the matrix has a high surface-to-volume ratio for culturing anchorage dependent cells. According to various embodiments, the matrix can be arranged or packed in a bioreactor in certain ways discussed here for uniform cell seeding and growth, uniform media perfusion, and efficient cell harvest.

According to one or more embodiments the cell culture substrate can be one according to the cell culture matrices and/or substrate materials disclosed in U.S. patent application Ser. Nos. 16/781,685; 16/781,723; 16/781,764; 16/781,807; 16/781,847; 16/781,883; and Ser. No. 16/765,722, all of which are incorporated herein by reference in their entireties.

According to some embodiments, a method of cell culturing is also provided using bioreactors with the matrix for bioprocessing production of therapeutic proteins, antibodies, viral vaccines, or viral vectors.

The cell culture substrates and bioreactor systems provided offer numerous advantages. For example, the embodiments of this disclosure can support the production of any of a number of viral vectors, such as AAV (all serotypes) and lentivirus, and can be applied toward in vivo and ex vivo gene therapy applications. The uniform cell seeding and distribution maximizes viral vector yield per vessel, and the designs enable harvesting of viable cells, which can be useful for seed trains consisting of multiple expansion periods using the same platform. In addition, the embodiments herein are scalable from process development scale to production scale, which ultimately saves development time and cost. The methods and systems disclosed herein also allow for automation and control of the cell culture process to maximize vector yield and improve reproducibility. Finally, the number of vessels needed to reach production-level scales of viral vectors (e.g., 10¹⁶ to 10¹⁸ AAV VG per batch) can be greatly reduced compared to other cell culture solutions.

Embodiments are not limited to the vessel rotation about a central longitudinal axis. For example, the vessel may rotate about an axis that is not centrally located with respect to the vessel. In addition, the axis of rotation may be a horizontal or vertical axis.

### Definitions

“Wholly synthetic” or “fully synthetic” refers to a cell culture article, such as a microcarrier or surface of a culture vessel, that is composed entirely of synthetic source materials and is devoid of any animal derived or animal sourced materials. The disclosed wholly synthetic cell culture article eliminates the risk of xenogeneic contamination.

“Include,” “includes,” or like terms means encompassing but not limited to, that is, inclusive and not exclusive.

“Users” refers to those who use the systems, methods, articles, or kits disclosed herein, and include those who are culturing cells for harvesting of cells or cell products, or those who are using cells or cell products cultured and/or harvested according to embodiments herein.

“About” modifying, for example, the quantity of an ingredient in a composition, concentrations, volumes, process temperature, process time, yields, flow rates, pressures, viscosities, and like values, and ranges thereof, or a dimension of a component, and like values, and ranges thereof, employed in describing the embodiments of the disclosure, refers to variation in the numerical quantity that can occur, for example: through typical measuring and handling procedures used for preparing materials, compositions, composites, concentrates, component parts, articles of manufacture, or use formulations; through inadvertent error in these procedures; through differences in the manufacture, source, or purity of starting materials or ingredients used to carry out the methods; and like considerations. The term “about” also encompasses amounts that differ due to aging of a composition or formulation with a particular initial concentration or mixture, and amounts that differ due to mixing or processing a composition or formulation with a particular initial concentration or mixture.

“Optional” or “optionally” means that the subsequently described event or circumstance can or cannot occur, and that the description includes instances where the event or circumstance occurs and instances where it does not.

The indefinite article “a” or “an” and its corresponding definite article “the” as used herein means at least one, or one or more, unless specified otherwise.

Abbreviations, which are well known to one of ordinary skill in the art, may be used (e.g., “h” or “hrs” for hour or hours, “g” or “gm” for gram(s), “mL” for milliliters, and “rt” for room temperature, “nm” for nanometers, and like abbreviations).

Specific and preferred values disclosed for components, ingredients, additives, dimensions, conditions, and like aspects, and ranges thereof, are for illustration only; they do not exclude other defined values or other values within defined ranges. The systems, kits, and methods of the disclosure can include any value or any combination of the values, specific values, more specific values, and preferred values described herein, including explicit or implicit intermediate values and ranges.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that any particular order be inferred.

It will be apparent to those skilled in the art that various modifications and variations can be made without departing from the spirit or scope of the disclosed embodiments. Since modifications, combinations, sub-combinations and variations of the disclosed embodiments incorporating the spirit and substance of the embodiments may occur to persons skilled in the art, the disclosed embodiments should be construed to include everything within the scope of the appended claims and their equivalents.

### Illustrative Implementations

The following is a description of various aspects of implementations of the disclosed subject matter. Each aspect may include one or more of the various features, characteristics, or advantages of the disclosed subject matter. The implementations are intended to illustrate a few aspects of the disclosed subject matter and should not be considered a comprehensive or exhaustive description of all possible implementations.

Aspect 1 pertains to a cell culture bioreactor comprising: a cell culture vessel comprising an interior reservoir configured to contain a cell substrate, an inlet fluidly connected to the reservoir, and an outlet fluidly connect to the reservoir; and a sensor system for measuring impedance within the reservoir, the sensor system comprising a pair of electrodes configured to be spaced apart from each other with at least a portion of the cell substrate disposed between the pair of electrodes, wherein each electrode of the pair of electrodes comprises a plurality of pores configured to allow at least one of cells and culture media to flow through the electrode.

Aspect 2 pertains to the cell culture bioreactor of Aspect 1, wherein the sensor system further comprises a plurality of pairs of electrodes, each pair of electrodes being configured to be spaced apart from each other with at least a portion of the cell substrate disposed between the pair of electrodes.

Aspect 3 pertains to the cell culture bioreactor of Aspect 2, wherein the plurality of pairs of electrodes are disposed in different regions of the reservoir.

Aspect 4 pertains to the cell culture bioreactor of Aspect 3, wherein the plurality of pairs of electrodes are spaced axially along a length of the reservoir, the plurality of pairs of electrodes comprising a pair of electrodes in at least two of a top region of the reservoir, a bottom region of the reservoir, and a central region of the reservoir.

Aspect 5 pertains to the cell culture bioreactor of Aspect 3, wherein the plurality of pairs of electrodes are spaced radially within a width of the reservoir, the plurality of pairs of electrodes comprising a pair of electrodes in at least two of a central region of the reservoir, an outer region of the reservoir, and a middle region of the reservoir.

Aspect 6 pertains to the cell culture bioreactor of Aspects 1-5, further comprising a cell substrate disposed within the reservoir and being configured for adhering cells thereto for cell culture.

Aspect 7 pertains to the cell culture bioreactor of Aspect 6, wherein the cell substrate comprises a structurally defined multi-layered substrate.

Aspect 8 pertains to the cell culture bioreactor of Aspect 7, wherein each layer of the multi-layered substrate comprises a physical structure and a porosity that are substantially regular and uniform.

Aspect 9 pertains to the cell culture bioreactor of Aspects 6-8, wherein the cell substrate comprises a substantially uniform porosity.

Aspect 10 pertains to the cell culture bioreactor of Aspects 6-9, wherein the cell substrate is configured for uniform fluid flow therethrough.

Aspect 11 pertains to the cell culture bioreactor of Aspects 6-10, wherein the reservoir is defined by a length and a width, the length extending from a first end of the reservoir adjacent to the inlet to a second end of the reservoir adjacent to the outlet, the substrate layers each having a width extending substantially across a width of the reservoir.

Aspect 12 pertains to the cell culture bioreactor of Aspects 6-11, wherein the structurally defined multi-layered substrate comprises a plurality of substrate disks stacked in the reservoir.

Aspect 13 pertains to the cell culture bioreactor of Aspect 12, wherein the stack of the plurality of substrate disks is configured to exhibit substantially uniform fluid flow across a width of each of the plurality of substrate disks.

Aspect 14 pertains to the cell culture bioreactor of Aspect 12, wherein structurally defined multi-layered substrate comprises a plurality of openings defining the porosity, the plurality of openings being arrayed in a regular or uniform pattern in each disk of the plurality of substrate disks.

Aspect 15 pertains to the cell culture bioreactor of Aspects 6-14, wherein the structurally defined multi-layered substrate comprises at least one of a molded polymer lattice, a 3D-printed lattice, and a woven substrate.

Aspect 16 pertains to the cell culture bioreactor of Aspect 6, wherein the cell substrate comprises a plurality of substrate layers, at least a portion of the plurality of substrate layers are not separated by a spacer material or barrier, or are in physical contact with each other.

Aspect 17 pertains to the cell culture bioreactor of any of the preceding Aspects 1-16, wherein the cell culture vessel is configured for cell culture media to flow continuously from the inlet toward the outlet during a cell culture period.

Aspect 18 pertains to the cell culture bioreactor of any of the preceding Aspects 1-17, wherein each electrode of the pair of electrodes comprises a substantially uniform porosity.

Aspect 19 pertains to the cell culture bioreactor of any of the preceding Aspects, wherein each electrode of the pair of electrodes comprises a physical structure and a porosity that are substantially regular and uniform.

Aspect 20 pertains to the cell culture bioreactor of any of the preceding Aspects, wherein each electrode of the pair of electrodes is configured for uniform fluid flow therethrough.

Aspect 21 pertains to the cell culture bioreactor of any of the preceding Aspects, wherein each electrode of the pair of electrodes extends across a width of the reservoir.

Aspect 22 pertains to the cell culture bioreactor of any of the preceding Aspects, wherein the sensor system is configured to measure a change in at least one of impendence and capacitance between the pair of electrodes over a period of time while culturing cells within the reservoir.

Aspect 23 pertains to the cell culture bioreactor of Aspect 22, wherein the sensor system is configured to monitor a proliferation of adherent cells in the cell substrate disposed at least between the pair of electrodes.

Aspect 24 pertains to the cell culture bioreactor of Aspect 22 or Aspect 23, wherein the sensor system is configured to predict a proliferation of adherent cells through the reservoir.

Aspect 25 pertains to the cell culture bioreactor of any of the preceding Aspects, further comprising a controller configured to receive signals from the sensor system, the controller comprising a microprocessor for analyzing the signals measured by the sensor system.

Aspect 26 pertains to the cell culture bioreactor of Aspect 25, wherein the controller comprises a set of instructions stored in memory which, when executed by the microprocessor, causes the controller to predict the proliferation of adherent cells in the cell substrate based on the signals detected by the sensor system.

Aspect 27 pertains to the cell culture bioreactor of any of the preceding Aspects, wherein the pair of electrodes comprises at least one of (1) a porous polymer structure and a conductive coating disposed on the porous polymer structure, and (2) a porous polymer structure comprising a conductive polymer.

Aspect 28 pertains to the cell culture bioreactor of Aspect 27, wherein the conductive coating comprises gold, or platinum.

Aspect 29 pertains to the cell culture bioreactor of Aspect 27 or Aspect 28, wherein the porous polymer structure is the same material as the cell substrate.

Aspect 30 pertains to the cell culture bioreactor of Aspects 27-29, wherein the porous polymer structure has the same structure, geometry, and/or porosity as the cell substrate.

Aspect 31 pertains to the cell culture bioreactor of any of the preceding Aspects, wherein the sensor system is configured to measure the impedance aseptically during a cell culture.

Aspect 32 pertains to the cell culture bioreactor of any of the preceding Aspects, wherein the sensor system is configured to measure impedance in real time during a cell culture.

Aspect 33 pertains to the cell culture bioreactor of any of the preceding Aspects, wherein the sensor system is an integral part of the cell culture vessel or the fixed bed.

Aspect 34 pertains to the cell culture bioreactor of Aspect any of the preceding Aspects, wherein the pair of electrodes are separated by a plurality of layers of the cell substrate.

Aspect 35 pertains to a method of monitoring biomass during a cell culture of cells in a bioreactor, the method comprising: culturing the cells on a fixed bed within the bioreactor using a cell culture medium perfused through the bioreactor; measuring at least one of impedance and capacitance across at least a portion of the fixed bed during the culturing of the cells.

Aspect 36 pertains to the method of Aspect 35, further comprising determining at least one of a density and proliferation of cells in the fixed bed.

Aspect 37 pertains to the method of Aspect 35 or Aspect 36, wherein the measuring comprises measuring the impedance or capacitance using a pair of electrodes that are spaced apart from each other with at least a portion of the fixed bed being disposed between the pair of electrodes.

Aspect 38 pertains to the method of any of Aspects 35-37, wherein the measuring the at least one of impedance and capacitance comprises taking multiple or continuous measurements over a period of time during a cell culture.

Aspect 39 pertains to the method of any of Aspects 35-38, wherein the measuring occurs in real time and aseptically.

Aspect 40 pertains to a fixed bed cell substrate for culturing adherent cells in a bioreactor, the fixed bed cell substrate comprising: a cell substrate configured for adhering cells thereto for cell culture; and an impedance sensor comprising a pair of electrodes that are spaced apart, wherein at least a portion of the cell substrate is disposed between the pair of electrodes.

Aspect 41 pertains to the fixed bed cell culture substrate of Aspect 40, wherein each electrode of the pair of electrodes comprises a plurality of pores configured to allow at least one of cells and culture media to flow through the electrode.

Aspect 42 pertains to the fixed bed cell culture substrate of Aspect 40 or Aspect 41, further comprising a plurality of impedance sensors, each of the plurality of impedance sensors comprising a pair of electrodes that are spaced apart from each other with at least a portion of the cell substrate disposed between the pair of electrodes.

Aspect 43 pertains to the fixed bed cell culture substrate of Aspect 42, wherein the plurality of pairs of electrodes are disposed in different regions of the fixed bed cell substrate.

Aspect 44 pertains to the fixed bed cell culture substrate of Aspects 40-43, wherein the cell substrate comprises a structurally defined multi-layered substrate.

Aspect 45 pertains to the fixed bed cell culture substrate of Aspect 44, wherein each layer of the multi-layered substrate comprises a physical structure and a porosity that are substantially regular and uniform.

Aspect 46 pertains to the fixed bed cell culture substrate of Aspects 40-45, wherein the cell substrate comprises a substantially uniform porosity.

Aspect 47 pertains to the fixed bed cell culture substrate of Aspects 40-46, wherein the cell substrate is configured for uniform fluid flow therethrough.

Aspect 48 pertains to the fixed bed cell culture substrate of Aspect 42, wherein the plurality of pairs of electrodes are spaced axially along a length of the fixed bed cell substrate, the plurality of pairs of electrodes comprising a pair of electrodes in at least two of a top region of the fixed bed cell substrate, a bottom region of the fixed bed cell substrate, and a central region of the fixed bed cell substrate.

Aspect 49 pertains to the fixed bed cell culture substrate of Aspect 42, wherein the plurality of pairs of electrodes are spaced radially within a width of the fixed bed cell substrate, the plurality of pairs of electrodes comprising a pair of electrodes in at least two of a central region of the fixed bed cell substrate, an outer region of the fixed bed cell substrate, and a middle region of the fixed bed cell substrate.

Aspect 50 pertains to the fixed bed cell culture substrate of Aspects 40-49, wherein the cell substrate comprises a plurality of layers of substrate in a stacked arrangement.

Aspect 51 pertains to the fixed bed cell culture substrate of Aspects 40-49, wherein the cell substrate comprises a cylindrical roll of substrate material.

Aspect 52 pertains to the fixed bed cell culture substrate of Aspect 51, wherein the cylindrical roll comprises a plurality of layers of cell substrate rolled together.

Aspect 53 pertains to the fixed bed cell culture substrate of Aspects 40-52, wherein each electrode of the pair of electrodes comprises a substantially uniform porosity.

Aspect 54 pertains to the fixed bed cell culture substrate of Aspects 40-53, wherein each electrode of the pair of electrodes comprises a physical structure and a porosity that are substantially regular and uniform.

Aspect 55 pertains to the fixed bed cell culture substrate of Aspects 40-54, wherein each electrode of the pair of electrodes is configured for uniform fluid flow therethrough.

Aspect 56 pertains to the fixed bed cell culture substrate of Aspects 40-55, wherein each electrode of the pair of electrodes extends across a width of the reservoir.

Aspect 57 pertains to the fixed bed cell culture substrate of Aspects 40-56, wherein the pair of electrodes comprises at least one of (1) a porous polymer structure and a conductive coating disposed on the porous polymer structure, and (2) a porous polymer structure comprising a conductive polymer.

Aspect 58 pertains to the fixed bed cell culture substrate of Aspect 57, wherein the conductive coating comprises gold, or platinum.

Aspect 59 pertains to the fixed bed cell culture substrate of Aspect 57 or 58, wherein the porous polymer structure is the same material as the cell substrate.

Aspect 60 pertains to the fixed bed cell culture substrate of Aspects 57-59, wherein the porous polymer structure has the same structure, geometry, and/or porosity as the cell substrate.

Aspect 61 pertains to a fixed bed bioreactor for cell culture comprising: a cell culture vessel comprising a reservoir configured to contain a cell substrate, an inlet fluidly connected to the reservoir, and an outlet fluidly connect to the reservoir; and a sensor within the reservoir, the sensor being configured to detect an electrical property of the cell culture within the reservoir, wherein the electrode is configured to detect the electrical property during an active cell culture.

Aspect 62 pertains to the fixed bed cell culture substrate of Aspect 61, wherein the sensor is configured to be disposed on top of, below, or within the cell substrate.

Aspect 63 pertains to the fixed bed cell culture substrate of Aspect 61 or Aspect 62, wherein the sensor is integrated into the cell substrate.

Aspect 64 pertains to the fixed bed cell culture substrate of Aspect 61, wherein the sensor is adhered to a sidewall of the reservoir.

Aspect 65 pertains to the fixed bed cell culture substrate of Aspects 61-64, further comprising a controller configured to receive signals from the sensor, the controller comprising a processor for analyzing the signals measured by the sensor.

Aspect 66 pertains to the fixed bed cell culture substrate of Aspect 65, wherein the property of the cell culture is a confluency of the cell culture or a culture quality of the cell culture.

Aspect 67 pertains to the fixed bed cell culture substrate of Aspects 61-66, further comprising a cell substrate disposed within the reservoir and being configured for adhering cells thereto for cell culture.

Aspect 68 pertains to the fixed bed cell culture substrate of Aspect 67, wherein the cell substrate comprises a structurally defined multi-layered substrate.

Aspect 69 pertains to the fixed bed cell culture substrate of Aspect 68, wherein each layer of the multi-layered substrate comprises a physical structure and a porosity that are substantially regular and uniform.

Aspect 70 pertains to the fixed bed cell culture substrate of Aspects 67-69, wherein the cell substrate comprises a substantially uniform porosity.

Aspect 71 pertains to the fixed bed cell culture substrate of Aspects 67-70, wherein the cell substrate is configured for uniform fluid flow therethrough.

Aspect 72 pertains to the fixed bed cell culture substrate of Aspects 67-71, wherein the reservoir is defined by a length and a width, the length extending from a first end of the reservoir adjacent to the inlet to a second end of the reservoir adjacent to the outlet, the substrate layers each having a width extending substantially across a width of the reservoir.

Aspect 73 pertains to the fixed bed cell culture substrate of Aspects 67-72, wherein the structurally defined multi-layered substrate comprises a plurality of substrate disks stacked in the reservoir.

Aspect 74 pertains to the fixed bed cell culture substrate of Aspect 73, wherein the stack of the plurality of substrate disks is configured to exhibit substantially uniform fluid flow across a width of each of the plurality of substrate disks.

Aspect 75 pertains to the fixed bed cell culture substrate of Aspect 73, wherein structurally defined multi-layered substrate comprises a plurality of openings defining the porosity, the plurality of openings being arrayed in a regular or uniform pattern in each disk of the plurality of substrate disks.

Aspect 76 pertains to the fixed bed cell culture substrate of Aspects 67-75, wherein the structurally defined multi-layered substrate comprises at least one of a molded polymer lattice, a 3D-printed lattice, and a woven substrate.

Aspect 77 pertains to the fixed bed cell culture substrate of Aspect 67, wherein the cell substrate comprises a plurality of substrate layers, at least a portion of the plurality of substrate layers are not separated by a spacer material or barrier, or are in physical contact with each other.

Aspect 78 pertains to the fixed bed cell culture substrate of Aspects 61-77, wherein the cell culture vessel is configured for cell culture media to flow continuously from the inlet toward the outlet during a cell culture period.

Aspect 79 pertains to the fixed bed cell culture substrate of Aspects 61-78, wherein the sensor is configured to maintain uniform fluid flow through the cell substrate.

Aspect 80 pertains to the fixed bed cell culture substrate of Aspects 61-79, wherein the sensor is configured to predict a proliferation of adherent cells through the reservoir.

Aspect 81 pertains to the fixed bed cell culture substrate of Aspect 65, wherein the controller comprises a set of instructions stored in memory which, when executed by the microprocessor, causes the controller to calculate the confluency of cells on the cell substrate.

Aspect 82 pertains to the fixed bed cell culture substrate of Aspects 61-81, wherein the sensor comprises one or more electrodes comprising at least one of a metal, a metal alloy, a conductive polymer, and a conductive polymer hydrogel.

Aspect 83 pertains to the fixed bed cell culture substrate of Aspect 82, wherein the one or more electrodes comprise gold, platinum, titanium, iridium, tungsten, or tantalum.

Aspect 84 pertains to the fixed bed cell culture substrate of Aspects 61-83, further comprising a plurality of sensors disposed in different locations within the reservoir, wherein the plurality of sensors is configured to detect cell culture homogeneity across the fixed bed of the cell substrate.

Aspect 85 pertains to the fixed bed cell culture substrate of Aspects 61-84, wherein the sensor is integrated onto a piece of the cell substrate such that the sensor does not interfere with the porosity of the cell substrate or fluid flow through the cell substrate.

Aspect 86 pertains to the fixed bed cell culture substrate of Aspects 61-85, wherein the sensor comprises a pair of impedance electrodes.

Aspect 87 pertains to the fixed bed cell culture substrate of Aspect 86, wherein the pair of impedance electrodes comprises interdigitated electrodes.

Aspect 88 pertains to the fixed bed cell culture substrate of Aspect 87, wherein the interdigitated electrodes are disposed on a same piece of cell substrate.

Aspect 89 pertains to the fixed bed cell culture substrate of Aspect 86, wherein the pair of electrodes comprises concentric impedance electrodes.

Aspect 90 pertains to the fixed bed cell culture substrate of Aspect 89, wherein the concentric impedance electrodes are disposed on a same side of a same piece of cell substrate.

Aspect 91 pertains to the fixed bed cell culture substrate of Aspects 61-85, wherein the sensor comprises circular cyclic voltammetry electrodes.

Aspect 92 pertains to the fixed bed cell culture substrate of Aspect 91, wherein the circular cyclic voltammetry electrodes are disposed separately on two different pieces of cell substrate.

Aspect 93 pertains to the fixed bed cell culture substrate of Aspect 91, wherein the circular cyclic voltammetry electrodes are disposed on opposite sides of a same piece of cell substrate.

Aspect 94 pertains to the fixed bed cell culture substrate of Aspects 61-85, wherein the sensor comprises a combination of impedance electrodes and cyclic voltammetry electrodes.

Aspect 95 pertains to the fixed bed cell culture substrate of Aspects 61-94, further comprising contact pads electrically connected to the sensor and configured to supply electrical power to the sensor.

Aspect 96 pertains to the fixed bed cell culture substrate of Aspect 95, wherein the contact pads are at least partially disposed outside of the reservoir.

Aspect 97 pertains to the fixed bed cell culture substrate of Aspects 61-85, wherein the sensor comprises a quartz crystal microbalance (QCM) sensor.

Aspect 98 pertains to the fixed bed cell culture substrate of Aspect 97, wherein the quartz crystal microbalance (QCM) sensor comprises a piezoelectric quartz crystal plate with electrodes evaporated onto both sides.

Aspect 99 pertains to the fixed bed cell culture substrate of Aspects 61-85, wherein the sensor comprises a radio-frequency impedance sensor or capacitor.

What is claimed:

1. A fixed bed bioreactor for cell culture comprising:
a cell culture vessel comprising a reservoir configured to contain a cell substrate, an inlet fluidly connected to the reservoir, and an outlet fluidly connect to the reservoir; and
a sensor within the reservoir, the sensor being configured to detect an electrical property of the cell culture within the reservoir,
wherein the electrode is configured to detect the electrical property during an active cell culture.

2. The fixed bed bioreactor of claim 1, wherein the sensor is integrated into the cell substrate.

3. The fixed bed bioreactor of claim 1, wherein the sensor is adhered to a sidewall of the reservoir.

4. The fixed bed bioreactor of claim 1, further comprising a cell substrate disposed within the reservoir and being configured for adhering cells thereto for cell culture.

5. The fixed bed bioreactor of claim 4, wherein the cell substrate comprises a structurally defined multi-layered substrate.

6. The fixed bed bioreactor of claim 5, wherein each layer of the multi-layered substrate comprises a physical structure and a porosity that are substantially regular and uniform.

7. The fixed bed bioreactor of claim 4, wherein the cell substrate comprises a substantially uniform porosity, and
wherein the cell substrate is configured for uniform fluid flow therethrough.

8. The fixed bed bioreactor of claim 4, wherein the cell substrate comprises a plurality of substrate layers, at least a portion of the plurality of substrate layers are not separated by a spacer material or barrier, or are in physical contact with each other.

9. The fixed bed bioreactor of claim 1, wherein the sensor is configured to predict a proliferation of adherent cells through the reservoir.

10. The fixed bed bioreactor of claim 1, further comprising a controller configured to receive signals from the sensor, the controller comprising a processor for analyzing the signals measured by the sensor, wherein the controller comprises a set of instructions stored in memory which, when executed by the microprocessor, causes the controller to calculate the confluency of cells on the cell substrate.

11. The fixed bed bioreactor of claim 1, wherein the sensor comprises one or more electrodes comprising at least one of a metal, a metal alloy, a conductive polymer, and a conductive polymer hydrogel.

12. The fixed bed bioreactor of claim 1, further comprising a plurality of sensors disposed in different locations within the reservoir,
wherein the plurality of sensors is configured to detect cell culture homogeneity across the fixed bed of the cell substrate.

13. The fixed bed bioreactor of claim 1, wherein the sensor comprises a pair of impedance electrodes.

14. The fixed bed bioreactor of claim 13, wherein the pair of impedance electrodes comprises interdigitated electrodes.

15. The fixed bed bioreactor of claim 14, wherein the interdigitated electrodes are disposed on a same piece of cell substrate.

16. The fixed bed bioreactor of claim 13, wherein the pair of electrodes comprises concentric impedance electrodes.

17. The fixed bed bioreactor of claim 1, wherein the sensor comprises circular cyclic voltammetry electrodes.

18. The fixed bed bioreactor of claim 1, wherein the sensor comprises a combination of impedance electrodes and cyclic voltammetry electrodes.

19. The fixed bed bioreactor of claim 1, further comprising contact pads electrically connected to the sensor and configured to supply electrical power to the sensor.

20. The fixed bed bioreactor of claim 19, wherein the contact pads are at least partially disposed outside of the reservoir.
